(19) United States
(12) Patent Application Publication
(10) Pub. No.: US 2022/0240791 A1
(43) Pub. Date: Aug. 4, 2022

(54) SYSTEMS AND METHODS FOR ESTIMATING BLOOD VELOCITY

(71) Applicant: Navix International Limited, Road Town (VG)

(72) Inventors: Shlomo BEN-HAIM, Milan (IT); Eyal Henri MADAR, Haifa (IL); Urit GORDON, Kiryat-Tivon (IL); Andrew ADLER, Ottawa (CA)

(73) Assignee: Navix International Limited, Road Town (VG)

(21) Appl. No.: 17/630,528

(22) PCT Filed: Aug. 4, 2020
(86) PCT No.: PCT/IB2020/057361

§ 371 Date: Jan. 27, 2022

Related U.S. Application Data
(60) Provisional application No. 62/882,526, filed on Aug. 4, 2019.

(51) Int. Cl. A61B 5/026; A61B 5/349; A61B 5/00; A61M 5/31

(52) U.S. Cl. CPC A61B 5/026; A61B 5/349; A61M 2205/3334; A61M 5/31; A61M 2230/04; A61B 5/7225

(57) ABSTRACT

Methods and systems are provided for estimating velocity of blood, flowing along a blood vessel at a blood flow direction, based on measurements made using a medical implement that resided in a portion of the blood vessel and comprised a first electrode and a second electrode. One of the disclosed methods include:
accessing voltage measurements measured at the first and second electrodes when a bolus of a fluid went through the portion of the blood vessel at the blood flow direction; wherein the voltage measurements were made at the first and the second electrodes; estimating a time that took the bolus to go from the first electrode to the second electrode based on the accessed voltage measurements; and estimating the velocity of the blood based on the estimated time and a distance known to exist along the medical implement between the first and second electrodes.

### RELATED APPLICATION

This application claims the benefit of priority of U.S. Provisional Patent Application No. 62/882,526 filed on Aug. 4, 2019, the contents of which are incorporated herein by reference in their entirety.

### BACKGROUND

The present invention, in some embodiments thereof, relates to intra-body medical measurements and, more specifically, but not exclusively, to systems and methods for estimating a velocity of blood.

Measurements of blood flow may be performed non-invasively, for example, using an ultrasound sensor to obtain Doppler data. Such Doppler data may detect blood flow problems in certain blood vessels in the body. Other measurements of blood flow are performed invasively, for example, using a catheter, for example, to compute fractional flow reserve (FFR) for evaluation of a stenosis in a blood vessel.

### SUMMARY

An aspect of some embodiments of the present invention includes a method of estimating velocity of blood, flowing along a blood vessel at a blood flow direction, based on measurements made using a medical implement that resided in a portion of the blood vessel and comprised a first electrode and a second electrode. In some embodiments, the method comprises:

accessing voltage measurements measured at the first and second electrodes when a bolus of a fluid went through the portion of the blood vessel at the blood flow direction; wherein the voltage measurements were made at the first and the second electrodes;

estimating a time that took the bolus to go from the first electrode to the second electrode based on the accessed voltage measurements; and

estimating the velocity of the blood based on the estimated time and a distance known to exist along the medical implement between the first and second electrodes.

In some embodiments, estimating the time comprises comparing voltage measurements made at first and second electrodes to obtain an estimate of a time that took the bolus to go from the first electrode to a the second electrode.

Optionally, said fluid has conductivity different from the conductivity of the blood by at least 20%.

In some embodiments, at least one of the voltage measurements is a measurement of voltage between two electrodes of the medical implement.

In any of the above embodiments, at least one of the voltage measurements may be a measurement of voltage between a body surface electrode and an electrode of the medical implement.

In any of the above embodiments, each one of the voltage measurements may be a measurement of voltage between two electrodes of the medical implement.

In any of the above embodiments, the method may further include detecting a peak in readings of each electrode, each corresponds to a respective peak time, and wherein time that took the bolus to go from the first electrode to the second electrode is computed based on a time difference between the peak time corresponding to the first electrode and the peak time corresponding to the second electrode.

Optionally, the peak is detected by cross-correlating the voltage measurements.

In any of the above embodiments, the method may further include obtaining a rough estimation of the blood flow rate, and controlling a bolus injector to inject the bolus into the blood vessel at a flow rate equal to the obtained rough estimation multiplied by a factor between ½ and 2.

Optionally, the rough estimation of the blood flow is non-invasively estimated using an external sensor.

In any of the above embodiments, the method may further include controlling a bolus injector to inject the bolus into the blood vessel, wherein the bolus is smaller than 0.5 milliliter (ml).

In any of the above embodiments, the method may further include controlling a bolus injector to inject the bolus within a time period smaller than 0.5 seconds.

In any of the above embodiments, velocity of blood may be defined in units of distance along the blood flow direction per units of time.

In any of the above embodiments, the bolus of fluid is synchronized for injection into the portion of the blood vessel during a selected portion of an ECG cycle.

Optionally, the time that took the bolus to go from the first electrode to the second electrode is within the selected portion of the ECG cycle.

Optionally, the velocity of blood is estimated from a plurality of boluses of fluid, each injected into the portion of the blood vessel during a different selected portion of the ECG cycle.

In some embodiments, the velocity of blood is estimated from a plurality of boluses of fluid, each injected into the portion of the blood vessel during a different portion of the ECG cycle, and a velocity of blood is computed based on respective passage times computed for each of the plurality of boluses, each passage time being time estimated to elapse between the bolus passing near the first electrode and the bolus passing near the second electrode.

In some embodiments, for at least one of the boluses, the time that took the bolus to go from the first electrode to the second electrode is a non-integer number of heart beats.

In some embodiments, the method includes providing different estimates of blood flow velocity to different selected ECG cycle portions.

In any of the above embodiments, the method may further include determining a blood flow defined in units of volume per time; and determining a diameter of the blood vessel based on the estimate of the velocity of the blood and the determined blood flow.

In any of the above embodiments, the method may further include diagnosing, according to the velocity of the blood, a narrowing of the blood vessel requiring treatment with a stent.

In any one of the above embodiments, the method may further include inserting a stent into a narrowing of the blood vessels detected according to an analysis of the velocity of the blood. In any of the above embodiments, the electrodes may be spaced apart along an axial axis of the medical implement.

In any of the above embodiments, a sheath along the medical implement includes at least one aperture for injection of the bolus of fluid, the at least one aperture located proximal to the electrodes such that in use blood flowing at the blood flow direction passes first near the aperture and then near the electrodes.

In any one of the above embodiments, the medical implement may include a catheter.

In any one of the above embodiments, a distance between a location of injection of the bolus of fluid along the medical implement and the first electrode is about 2 to 6 centimeters.

An aspect of some embodiments of the invention includes a system for estimating velocity of blood, flowing along a blood vessel at a blood flow direction, utilizing data received via a catheter that resided in a portion of the blood vessel and comprised electrodes. In some embodiments the system comprises at least one hardware processor executing a code for: accessing voltage measurements made at the electrodes when a bolus of a fluid goes through the portion of the blood vessel at the blood flow direction;

comparing the accessed voltage measurements made at the electrodes to obtain an estimate of a time that took the bolus to go from a first one of the electrodes to a second one of the electrodes; and providing the estimate based on the comparison and a distance known to exist along the catheter between the first and second electrodes.

In some embodiments, the system further includes code for controlling a bolus injector to inject the bolus into the blood vessel.

Any of the above systems may further include a bolus injector set to inject the bolus into the blood vessel.

Any of the above systems may further include an ECG device that outputs an indication of an ECG signal obtained from ECG sensors sensing the patient, wherein the bolus of fluid is synchronized for injection into the portion of the blood vessel during a selected portion of an ECG cycle.

Optionally, the ECG device outputs the indication to the at least one hardware processor or to a memory accessible by the at least one hardware processor, and the at least one hardware processor controls the injector based on signals received from the ECG device.

Any one of the above systems may further include code for controlling a bolus injector to inject the bolus into the portion of the blood vessel during a selected portion of an ECG cycle obtained from an ECG device that outputs an indication of an ECG signal obtained from ECG sensors sensing the patient.

Any one of the above systems may further include a sheath along the medical implement including at least one aperture for injection of the bolus of fluid, the at least one aperture located proximal to the electrodes such that in use blood flowing at the blood flow direction passes first near the aperture and then near the electrodes.

In any one of the above systems, the medical implement may include a catheter.

Any one of the above systems may further include a current source to apply a current to at least one of the electrodes, an ampere meter for measuring the resulting current between the electrodes, and a voltmeter for measuring voltage between the electrodes when the current is applied.

An aspect of some embodiments of the invention includes a computer program product for estimating velocity of blood, flowing along a blood vessel at a blood flow direction, utilizing a catheter that resides in a portion of the blood vessel and comprises electrodes. In some embodiments, the computer program product includes a non-transitory memory storing thereon code for execution by at least one hardware process, the code including instructions for:

accessing voltage measurements made by the electrodes when a bolus of a fluid goes through the portion of the blood vessel at the blood flow direction, comparing voltage measurements made at two of the electrodes to obtain an estimate of a time that took the bolus to go from a first one of the electrodes to a second one of the electrodes; and dividing the estimate obtained from the comparison by a distance known to exist along the catheter between the first and second electrodes to obtain the estimate of the velocity of the blood.

An aspect of some embodiments of the invention includes a computer program product for estimating velocity of blood, flowing along a blood vessel at a blood flow direction, utilizing a catheter that resides in a portion of the blood vessel and comprises electrodes. The computer program product includes a non-transitory memory storing thereon code for execution by at least one hardware processor, the code including instructions for carrying out a method as described in any method described above.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIG. 1A is a flowchart of a method for selecting a patient and diagnosing and/or treating the selected patient by estimating velocity of blood, flowing along a blood vessel at a blood flow direction, utilizing a medical implement that resides in a portion of the blood vessel and comprises a first electrode and a second electrode, in accordance with some embodiments of the present invention;

FIG. 1B is a flowchart of a method of estimating velocity of blood, flowing along a blood vessel at a blood flow direction, utilizing a medical implement that resides in a portion of the blood vessel and comprises a first electrode and a second electrode, in accordance with some embodiments of the present invention;

FIG. 1C is a flowchart of a method of treating a patient based on estimating velocity of blood flowing in a blood vessel, in accordance with some embodiments of the present invention;

FIG. 2A is a block diagram of components of a system for estimating velocity of blood, in accordance with some embodiments of the present invention;

FIG. 2B is a block diagram of components of another embodiment of the system for estimating velocity of blood, in accordance with some embodiments of the present invention;

FIG. 3 is a schematic of a catheter used to measure blood velocity in the coronary arteries of a pig during in vivo experiments, in accordance with some embodiments of the present invention;

FIG. 4 is a schematic of an experimental setup for evaluating an in vitro blood velocity estimation process;

FIG. 5 includes graphs depicting flow measurements received during in vitro experiment (upper graph) and the in vivo experiment (lower graphs), according to embodiments of the present invention;

FIG. 6 includes graphs depicting time development of voltage on two electrodes during a single bolus, useful for estimating blood velocity according to embodiments of the present invention; and

FIG. 7 includes a graph depicting change of dielectric contrast agent velocity estimated for contrast agent in a tank due to different pump rates (on the left), and a graph depicting changes in blood velocity measured in a porcine before and after administration of nitroglycerine.

### DETAILED DESCRIPTION

As used herein, the terms medical implement refers to any device, tool, instrument, or appliance configured for intra-body (e.g., intra vascular) usage for medical purposes, such as exploration, injection or withdrawal of fluids, keeping a passage open, or facilitating the use of another medical implement. Non limiting examples to medical implements include guidewires, protection sheaths, implants and catheters, including but not limited to micro catheters, catheter probes, and ablation catheters. Examples or embodiments described herein as carried out with catheters may also be practiced with other medical implements.

An aspect of some embodiments of the present invention relates to systems, methods, apparatuses, and/or code instructions (i.e., stored on a memory and executable by one or more hardware processors) for estimating velocity of blood flowing along a blood vessel of a patient at a blood flow direction. The velocity of the blood is estimated utilizing a medical implement, optionally a catheter that resides in a portion of the blood vessel during the measurement. The medical implement includes at least two spaced apart electrodes located along a long axis of the medical implement. A bolus of fluid, injected by an injector at the blood flow direction, goes through the portion of the blood vessel and passes by the two electrodes. As the bolus of fluid goes through the portion of the blood vessel, voltage measurements made at the two electrodes are received. Changes in voltage measured at the electrodes are triggered by differences in electrical properties (e.g., conductivity) of the bolus of fluid relative to the electrical properties of blood, as the bolus of fluid travels along the blood vessel in the blood flow direction. The bolus of fluid is first in proximity to one electrode located proximal to the source of the bolus of fluid, and following a time interval when the bolus of fluid is carried by the blood in the blood flow direction, the bolus of fluid is in proximity to the second electrode located more distally to the source of the bolus of fluid. A time that it took the bolus to go from the first electrode to the second electrode (also referred herein as a passage time) is estimated, and blood velocity is estimated based on this time, e.g., by dividing a known distance between the two electrodes by the estimated time. The estimated velocity of blood may be analyzed for diagnosing and/or treating the patient. For example, the velocity of blood may be analyzed to determine whether a stenosis in the blood vessel is clinically significant or not, and/or if such a stenosis is to be treated, for example, by insertion of a stent.

Optionally, the injection is automatic, for example, the injector may be coupled to an ECG device that obtains ECG signals of the patient, and the injector may be controlled to inject the bolus at selected portion(s) of the heart beat cycle. Optionally, the injector is controlled to inject the bolus at multiple different parts of the heart beat cycle. Respective velocities of blood flow corresponding to the different parts of the heartbeat cycles may be computed and aggregated (e.g., averaged) to clean the results from the effects of the specific injection time.

Optionally, the passage time is estimated by comparing voltage measurements made at the first (i.e., proximal) and second (i.e., distal) electrodes to obtain an estimate of a time that it took the bolus to go from the first electrode to the second electrode. The velocity of the blood is estimated based on the estimated passage time and a distance known to exist along the catheter or other medical implement between the first and second electrodes.

Optionally, the difference in conductivity between the bolus of fluid and the blood is between about 10% and about 30%, for example, about 20%.

It is noted that at least some implementations of the systems, methods, apparatus, and/or code instructions described herein are not necessarily limited to blood vessels, but may be used to evaluate fluid flow in other body fluids, for example, blood flow in a synthetic graft (e.g., for dialysis patients), and urine flow in ureters.

At least some implementations of the systems, methods, apparatus, and/or code instructions described herein address the technical problem of estimating velocity of blood flowing through a blood vessel, for example, a coronary artery, a renal artery, or a carotid artery. The velocity of flowing blood may be used, for example, to determine whether the blood vessel is to be treated to increase the diameter thereof, for example, by insertion of a stent, balloon expansion of the vessel, and/or ablation of renal nerves to relax the renal artery. The velocity of flowing blood may be used to determine whether a blood vessel has been successfully treated or required additional treatment.

Angiography has become the standard practice for diagnosis and treatment of stenosis, for example, in the coronary arteries, carotid artery, renal artery, and other blood vessels in the body. Angiography with visual assessment alone has limitations in determining the severity and hemodynamic significance of lesions, especially in intermediate stenosis, where it is uncertain if a stent (or other treatment) is needed. Revascularization decisions may be based on the presence of ischemia. Not every stenosis, however, even if perceived as ‘severe’, causes ischemia. Equally important, lesions that do not appear severely stenotic may cause ischemia at times and may hence benefit from revascularization. Angiography in such cases is incomplete without assessment of ischemia.

Fractional Flow Reserve (FFR) and Instantaneous Wave-Free (iFR) are minimally-invasive diagnosis techniques used in conjunction with angiography to assess the physiology of lesions in order to guide decisions on whether or not to revascularize intermediate lesions. Yet, despite these great technological advances, current utilization of existing methods has remained fairly low (about 20% in the United States). Barriers to uptake include added procedural time (to perform the measurements), patient discomfort, increased clinical risks (e.g., use of vasodilators), and additional radiation exposure.

At least some implementations of the systems, methods, apparatus, and/or code instructions described herein estimate velocity of blood based on an injected substance and measurement by electrodes located distally to each other along a direction of the blood flow. The approaches described herein may provide technical advantages over FFR and iFR, for example, in that additional radiation exposure is not required (since impedance measurements of the blood with bolus may be done without additional radiation based imaging), patient discomfort and/or clinical risk may be low (such as when using saline and/or dextrose aqueous solution and using standard catheters and/or sheaths, in contrast to FFR that requires injection of a vasodilator and iFR that requires injection of a hyperemic agent, which pose a risk to patients) and/or procedure time may not be significantly increased (since measurements may be performed within a relatively short amount of time).

The approaches described herein are in contrast to yet other approaches that attempt to estimate changes in the diameter of the blood vessel by performing impedance measurements before and after a treatment procedure and comparing the post-treatment measurements to the pre-treatment measurements. Such approaches are not based on measuring injected substances to estimate blood velocity. In another example, occlusion quality is predicted based on an assessment of pulmonary vein occlusion by using injection of an impedance-modifying agent and evaluation of changes in impedance measurements recorded by an electrode located distal to an occlusion element of the treatment device used to inject the impedance-modifying agent. Such approaches are unable to estimate velocity of the blood based on an injected substance and measurement by electrodes located distally to each other along a direction of the blood flow.

At least some implementations of the systems, methods, apparatus, and/or code instructions described herein improve the technology of estimating velocity of blood flowing through a blood vessel. In at least some implementations the improvement is in terms of measuring a velocity of the blood in the blood vessel which may vary during very short periods of time, for example, during different phases of a single cardiac cycle corresponding to different patterns of the ECG signal where velocity may significantly vary between the different phases of the ECG signal. The improvement may be, for example, in terms of relatively higher accuracy of the measured velocity and/or flow in comparison to standard approaches. The improvement may be, for example, in terms of computation of a diameter of the vessel based on the estimated velocity. The vessel diameter can be estimated based on the blood velocity, carried out as disclosed herein, and blood flow (in volume per time units), which may be measured as known in the art. The estimation of the diameter of the vessels may be used to determine whether to treat the vessel and/or whether the treatment is successful and/or whether additional treatment is required.

At least some implementations of the systems, methods, apparatus, and/or code instructions described herein improve the medical treatment of blood vessels of a patient. The process of estimating velocity of blood flowing through a blood vessel may provide more accurate data for determining whether the blood vessels requires treatment or not. The process of estimating velocity of blood flowing through a blood vessel may be performed without necessarily administering additional radiation to the patient, for example, fluoroscopic imaging of a dye injected into the blood vessel.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

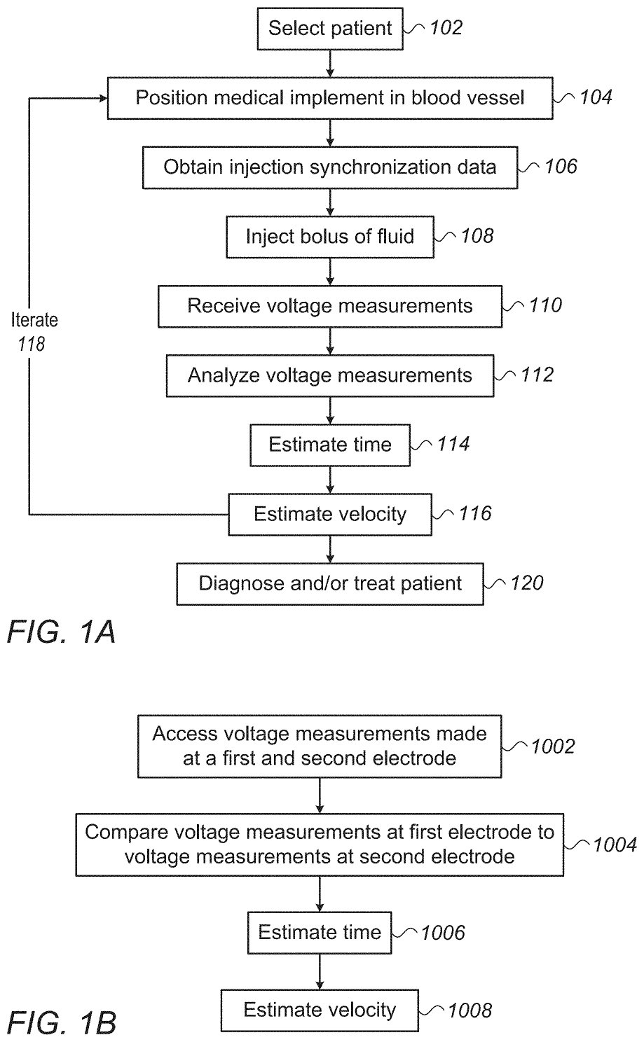

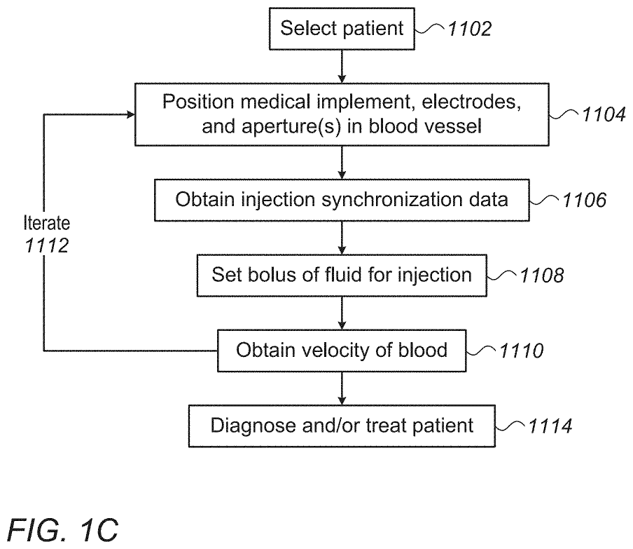

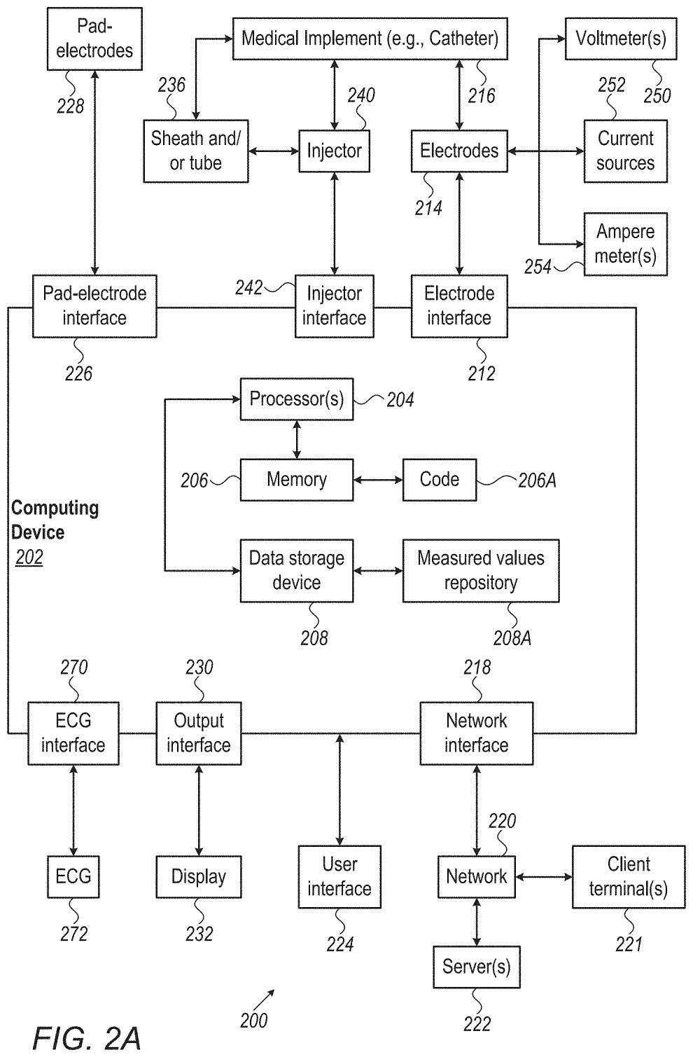

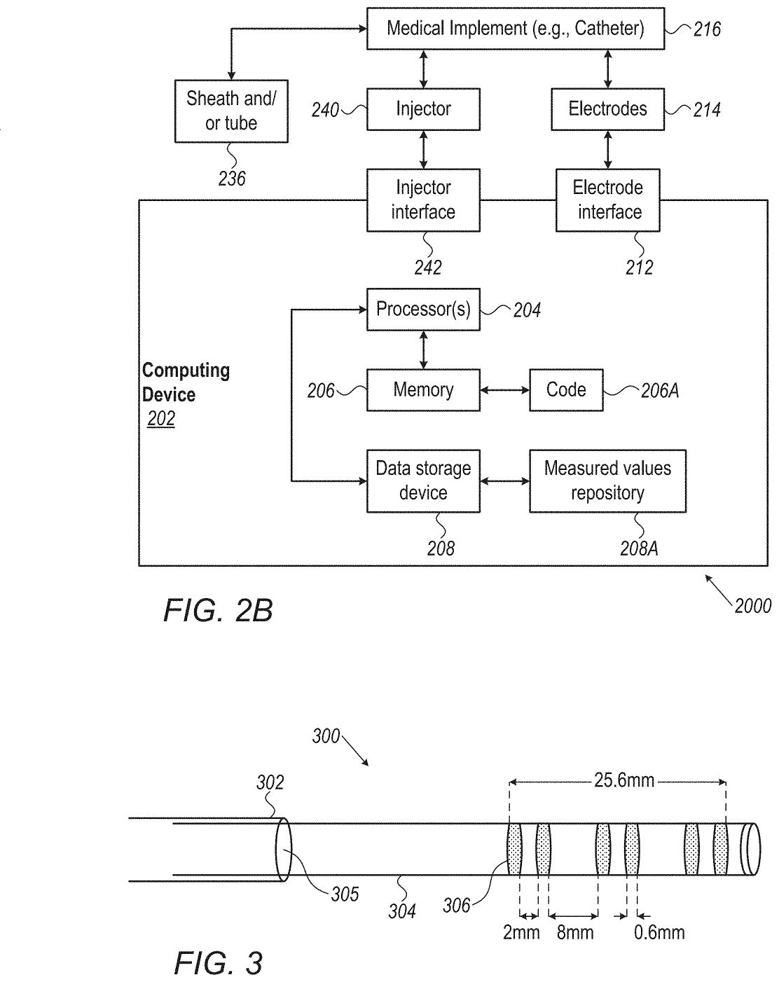

Reference is now made to FIG. 1A, which is a flowchart of a method for estimating velocity of blood, flowing along a blood vessel at a blood flow direction, utilizing a medical implement that resides in a portion of the blood vessel and comprises a first electrode and a second electrode, in accordance with some embodiments of the present invention. Reference is also made to FIG. 2A, which is a block diagram of components of a system 200 for estimating velocity of blood, flowing along a blood vessel at a blood flow direction, in accordance with some embodiments of the present invention. System 200 may implement the acts of the method described with reference to FIG. 1A, 1B, or 1C, optionally by a hardware processor(s) 204 of a computing device 202 executing code instructions 206A stored in a memory 206. Reference is now also made to FIG. 2B, which is a block diagram of components of another embodiment 2000 of a system for estimating velocity of blood. FIGS. 2A and 2B may include optional parts. For example, the system of FIG. 2B lacks some of the components of FIG. 2A to illustrate the optionality of the lacking parts. Parts of FIG. 2B common with FIG. 2A are described with reference to FIG. 2A.

Computing device 202 may be implemented as, for example, a client terminal, a server, a computing cloud, a virtual server, a virtual machine, a radiology workstation, a workstation installed within a catheterization laboratory, a mobile device, a desktop computer, a thin client, a Smartphone, a Tablet computer, a laptop computer, a wearable computer, glasses computer, and a watch computer.

Multiple architectures of system 200 based on computing device 202 may be implemented. For example, computing device 202 may be implemented as an existing device (e.g., client terminal) having software (e.g., code 206A) that performs one or more of the acts described with reference to FIG. 1A, for example, code 206A is installed on a computer conventionally existing in a catheterization/interventional lab. In another implementation, computing device 202 may be implemented as a dedicated device, having software (e.g., code 206A) installed thereon. In another exemplary implementation, computing device 202 storing code 206A may be implemented as one or more servers, for example, network server, web server, a computing cloud, a virtual server, a radiology server, an interventional laboratory server, that provides services based on one or more of the acts described with reference to FIG. 1A to one or more client terminals 221 over network 220. Client terminal 221 may be, in some embodiments, a terminal located remotely from computing device 202, for example, an interventional/catheterization laboratory client having access to computing device 202 acting as a server. In such as implementation, for example, remotely obtained sets of impedance measurements are transmitted from respective client terminals 221 to computing device 202 over network 220 for computation of the blood velocity and/or flow. The computed blood velocity and/or flow is transmitted from computing device 202 over network 220 to the respective client terminal 221, for example, for presentation on a display associated with the respective client terminal 221.

Hardware processor(s) 204 may be implemented for executing code 206A for implementing the acts of the method described with reference to FIG. 1A. In some embodiments, hardware processor(s) 204 may be implemented as a central processing unit(s) (CPU), a graphics processing unit(s) (GPU), field programmable gate array(s) (FPGA), digital signal processor(s) (DSP), and/or application specific integrated circuit(s) (ASIC). Processor(s) 204 may include one or more processors, which may be homogenous or heterogeneous, which may be arranged for parallel processing, as clusters and/or as one or more multi core processors.

Memory 206 stores code instructions executable by processor(s) 204. Memory 206 may be for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 206 stores code 206A.

Computing device 202 may include an electrode interface 212 for communicating with multiple electrodes 214 located on a distal end portion of medical implement 216. Exemplary electrode arrangements are described herein. The blood velocity and/or flow is computed according to data outputted by electrodes 214, as described herein. In some embodiments, impedance measurements used to compute blood flow and/or velocity are obtained using pad-electrodes 228. Pad-electrodes 228 may be controlled via a pad-electrode interface 226. Pad-electrodes 228 are positioned externally to the body of the patient for example, on the skin of the patient, and/or in the bed supporting the patient during the intervention.

In some embodiments, impedance measurements of the injected substance used for estimating blood velocity are obtained by electrodes 214 on medical implement 216 by applying a current to one electrode 214 using a current source 252, measuring voltages on each of the electrodes 214 using a respective voltmeter 250, and measuring the resulting current between electrodes 214 using an ampere meter 254. Measuring impedance between two electrodes 214 that both transmit to a pad electrode 228 is described, for example, with reference to International Patent Application No. PCT/IL2019/050501, titled “MEASURING ELECTRICAL IMPEDANCE, CONTACT FORCE, AND TISSUE PROPERTIES”, filed May 6, 2018, and published as WO2019/215721.

In some embodiments, computing device 202 is in communication with a bolus injector 240, optionally via an injector interface 242. Injector interface 242 may be implemented on computing device 202 and/or at the bolus injector 240. Injector interface 242 may be comprise, for example, one or more of: a wire connection, a wireless connection, a software interface (e.g., SDK, API), a virtual interface, a network interface, and a local bus. The bolus outputted by injector 240 may be directed into the blood vessel through a sheath and/or tube 236 that includes one or more apertures for exit of the injected bolus. Sheath 236 may be associated with medical implement 216, for example, sheath 236 is integrated with medical implement 216, for example, defining a channel within medical implement 216. In another example, sheath 236 sheathes medical implement 216, but ends proximally to at least two of the electrodes of medical implement 216. The contrast agent may be injected via a narrow space between the sheath and the catheter, and gets into the blood when the sheath ends, taken by the blood flow from one electrode to another. Injector 240 may be implemented, for example, as a mechanism designed to inject a bolus automatically based on instructions generated by computing device 202, semi-automatically and/or manually, for example, operated by an operator optionally as directed by instructions generated by computing device 202 that are presented on a display.

Computing device 202 may include an output interface 230 for communicating with a display 232, for example, a screen or a touch screen. Optionally, the computed blood flow and/or velocity is presented on display 232. Other data may be presented on display 232, for example, instructions to manually operate bolus injector 240, and/or an indication of status of automatic operation of bolus injector 240.

Optionally, computing device 202 includes a network interface 218, for communicating with server(s) 222 and/or client terminal(s) 221 over a network 220, for example, to obtain code 206A such as an updated version thereof, and/or transmit the computed blood velocity and/or flow to server(s) 222. Network interface 218 may be implemented as, for example, one or more of, a network interface card, a wireless interface to connect to a wireless network, a physical interface for connecting to a cable for network connectivity, a virtual interface implemented in software, network communication software providing higher layers of network connectivity, and/or other implementations.

Network 220 may be implemented as, for example, the internet, a local area network, a virtual network, a wireless network, a cellular network, a local bus, a point to point link (e.g., wired), and/or combinations of the aforementioned.

Optionally, a user interface 224 is in communication with computing device 202. User interface 224 may include a mechanism for the user to enter data, for example, a touch screen, a mouse, a keyboard, and/or a microphone with voice recognition software. In some embodiments, the user may enter data via a graphical user interface (GUI) presented on display 232, where the GUI acts as user interface 224.

Optionally, computing device includes an ECG interface 270 that communicates with an ECG machine 272. ECG machine 272 may output an indication of an ECG signal sensed by ECG sensors (e.g., electrodes) positioned on the patient. The ECG readings obtained by ECG machine 272 may be used to select times for injection of the bolus by injector 240, for example, to estimate blood velocity at different phases of the cardiac cycle, as described herein.

It is noted that one or more interfaces 218, 212, 226, 230, 242, and 270 may be implemented, for example, as a physical interface for example, cable interface, wireless interface, network interface, and/or as a virtual interface for example API, SDK. The interfaces may each be implemented separately, or multiple (e.g., a group or all) interfaces may be implemented as a single interface.

Processor 204 may be coupled to one or more of memory 206, data storage device 208, and interfaces 218, 212, 226, 230, 242, 270.

Optionally, computing device 202 includes data storage device 208, for example, measured values repository 208A for storing data collected from multiple electrodes 214 over a time interval, which is processed to compute the blood velocity and/or flow, as described herein. Data storage device 208 may be implemented as, for example, a memory, a local hard-drive, a removable storage device, an optical disk, a storage device, and/or as a remote server and/or computing cloud (e.g., accessed using a network connection).

It is noted that computing device 202 may include one or more of the following components: processor(s) 204, memory 206, data storage device 208, and interfaces 218, 212, 226, 230, 234, 242, and 270 for example, as a stand-alone computer, as a hardware card (or chip) implemented within a current computer, for example, catheterization laboratory computer, and/or as a computer program product loaded within the current computer.

Returning now back to FIG. 1A, at 102, a patient is selected for measurement of blood velocity in a portion of a blood vessel. The selection criteria used for selection of patients for measurement of velocity of blood in the blood vessel may be the same or similar to existing selection criteria for evaluation of patients for treatment of stenotic regions, for example, criteria for selecting patients for evaluation using FFR.

The blood vessel where the blood velocity is measured may include and/or be susceptible to a stenosis, which reduces the blood flow through the vessel. The blood vessel may be, for example, a coronary artery, a left anterior descending (LAD) artery, a carotid artery, and a femoral artery. The patient may be selected for evaluation of the clinical significance of the stenotic lesion, such as to determine whether the stenosis clinically reduces blood flow or not. The clinical significance of the stenosis may be evaluated, for example, by comparing the blood velocity before and after the lesion, for example, according to a requirement, such as a threshold and/or range. At 104, a medical implement, optionally a distal portion of a catheter including the at least two electrodes and optionally the aperture(s) through which the bolus is injected, is positioned within a portion of the blood vessels.

Different positioning arrangements are possible. In one example, the distal end of the catheter, the two electrodes, and the aperture are positioned on one side (i.e., distal or proximal) to a stenotic lesion in the blood vessel, for example, to compare the velocity of blood flow before and after the lesion. In another example, the aperture and at least one of the electrodes are proximally to the lesion, and at least one of the other electrodes distally to the lesion, to measure blood velocity through the lesion.

The catheter includes at least two spaced apart electrodes, positioned approximately along the direction of flow of blood, such that blood first reaches a first electrode (e.g., located proximally) before reaching a second electrode (e.g., located distally). The electrodes may be positioned spaced apart along an axial axis of the catheter.

The catheter may be, for example, an ablation catheter and/or other electrophysiological multi-electrode catheter. The electrodes in the catheter, may be used to measure velocity of the blood as described herein, in addition to their original design for ablation and/or performing other electrophysiological tasks.

As used herein, the reference to the first electrode (i.e., proximal) and second electrode (i.e., distal) may refer to each pair of the electrodes, and not necessarily to a single electrode pair. That is, the descriptors “first” and “second” and “distal” and “proximal” electrode is in comparison to the other electrode of the same pair, and not necessarily to the most proximal or most distal electrode on the catheter or other medical implement.

The medical implement includes at least two electrodes, for example, 3, 4, 5, 6, 8, 10, 20, or other number.

Referring now back to FIG. 1A, the fluid bolus may be, for example, a saline solution with electrical conductivity significantly higher or lower than that of blood. In another example, the fluid bolus may be a dextrose solution with electrical conductivity significantly lower than that of blood. The difference between the electrical conductivity of the bolus solution and the blood should be as large as possible, to provide large electrical effect with small volume of saline, but not so large as to adversely affect the patient. For example, as blood has typically electrical conductivity similar to that of a 1% saline solution, the bolus may be of concentration of 2%-3% saline or 0.3% to 0.5% saline, but good results are harder to obtain, if at all obtainable, with boluses of intermediate concentrations, e.g., between 0.5% to 2%. The aperture(s) are positioned upstream relative to the electrodes of the catheter, and positioned proximal to the electrodes, such that when blood is flowing in the blood flow direction, the blood first passes near the aperture, and carries the injected saline towards and past the electrodes, first passing by more proximal electrodes, and then passing by more distal electrodes.

Referring now back to FIG. 1A, optionally, a distance between a location of injection of the bolus of fluid along the catheter and the first electrode is about 2 to 5 centimeters, or about 1-4 centimeters, or about 3-6 centimeters, or other values and/or ranges. In some embodiments, distances of less than about 2 cm are preferred, for example, between about 1 cm and about 2 cm. The distance is selected to be far enough from the first (i.e., proximal) electrode to enable sufficient mixing of the bolus with the blood in the blood vessel prior to reaching the first electrode. When the bolus is not sufficiently mixed with the blood, the blood contacting the electrode might not have bolus within, resulting in a false measurement in which the voltage indicates no bolus while bolus is actually present in proximity to the electrode. When the distance selected is too far, the bolus may be spread out over a large distance along the length of the blood vessel by the flowing blood, reducing the impact of the presence of bolus on locally changing the conductivity of the blood, and making it more difficult and/or less accurate to detect the peak (or drop) in voltage for computation of the velocity of the blood.

At 106, injection synchronization data may be obtained. The injection synchronization data are used to set parameters of the injector for injection of the bolus, for example, timing of the injection and/or speed of the injected bolus.

The injection synchronization data may be automatically obtained by the computing device from the ECG machine, for automated control of the injector, as described herein. The injection synchronization data may be obtained from a sensor that provides a rough estimation of the blood velocity, and used to control the injector. The rough estimation of blood velocity may be manually obtained (e.g., using Doppler) and manually entered into the computing device and/or the injector for setting the speed of the injection, as described herein.

Optionally, the injection synchronization data includes a rough estimation of the blood velocity and/or flow rate within the blood vessel. The estimation of the blood velocity and/or flow rate may be obtained using invasive and/or non-invasive approaches, optionally standard approaches. For example, the blood velocity and/or flow in the blood vessel may be non-invasively estimated using an external sensor, for example, Doppler ultrasound. Alternatively, when an estimation of blood velocity and/or flow has already been determined as described herein at least one time for the blood vessel, the estimation of the blood velocity and/or flow rate is according to the previously estimated blood velocity and/or flow.

When the rough estimation of blood flow and/or velocity rate cannot be measured in the blood vessel (e.g., no sensor is available, no time to measure it), the rough estimation may be obtained, for example, from a table of empirical measurements performed in similar blood vessels of sample patients and/or a table of estimated values according to blood vessel and/or other mathematical estimation, for example, in the range of about 150-350 millimeters (mm) per second, optionally about 220 mm/second or other values in the range.

Optionally, the injection synchronization data includes an indication of the ECG cycle, which may be obtained from an ECG machine. Blood velocity may change according to different stages of the ECG cycle, for example, relatively higher during systole and relatively lower during diastole. Optionally, the injector is controlled to inject the bolus at multiple different parts of the heartbeat cycle corresponding to defined portions of the ECG signal. Each respective bolus injection may make more than the selected portion of the ECG signal, for example, more than half a cycle. Respective velocities of blood flow corresponding boluses injected at different parts of the heartbeat cycles may be computed and aggregated (e.g., averaged) to clean the results from the effects of the specific injection time.

At 108, the bolus of fluid is inserted into the blood vessel. The bolus of fluid may be injected into the blood vessel via the aperture of the sheath and/or tube, proximal to the electrodes of the catheter.

Optionally, the bolus of fluid includes saline of a selected concentration according to a selected conductivity, for example 3% saline or 7% saline or other selected values, for example, as described in the Examples section below. Alternatively or additionally, the bolus of fluid includes other fluid(s) and/or materials (e.g., dextrose water solution optionally at a weight/volume concentration of 5%), to obtain a target conductivity. The bolus of fluid may be selected to have a higher or lower conductivity than the blood in the blood vessel.

The bolus of fluid may be automatically injected by an injector device, optionally controlled by instructions generated by the computing device. Alternatively, the bolus of fluid may be manually injected.

The bolus injector may be controlled (e.g., automatically and/or manually) to inject a volume of bolus that is smaller than about 2 milliliter (ml), or 1 ml, or 0.5 ml, 0.3 ml, or about 0.1 ml, or about 0.05 ml, or within the range of about 0.2-1 ml, or about 0.1-0.5 ml, or 0.3 ml-2 ml, or other values.

The bolus injector may be controlled (e.g., automatically and/or manually) to inject the bolus within a time period smaller than about 0.5 seconds, or about 0.3 seconds, or about 0.1 second, or about 0.05 seconds, or about 0.02, or about 0.01, or other values.

Optionally, the injected fluid has conductivity that is different from the conductivity of the blood by at least about 10%, or 20%, or 30%, or other values. The difference in conductivity is selected to enable detection of the bolus within the blood vessel, as the bolus is carried by the blood, and travels in proximity to the electrodes of the catheter.

Optionally, the injection of the bolus is according to the injection synchronization data, for example, synchronized with the injection synchronization data, and/or controlled according to the injection synchronization data.

Optionally, the bolus injector is controlled to inject the bolus into the blood vessel at a flow rate equal to the obtained rough estimation of blood flow multiplied by a factor between ½ and 2, for example, approximately equal to the rough estimation of blood flow. The injection of the bolus based on the rough estimation of blood flow may reduce measurement errors arising from a mismatch between the injection flow and the blood flow.

Optionally, the bolus of fluid is synchronized for injection into the portion of the blood vessel during the selected portion of an ECG cycle, for example, to the QRS complex. Synchronizing the bolus of fluid with a selected portion of the ECG cycle improve repeatability and/or accuracy of the results. In some embodiments, the measurement of the blood velocity at the same corresponding portion of the ECG cycle may be repeated several times. The multiple measurements may be aggregated (e.g., averaged) to obtain a more clean signal (e.g., higher signal to noise ratio) than a single measurement. When the bolus is long enough to be spread along a substantial portion of the cardiac cycle, or even more than an entire cardiac cycle, for example, 1.5, 2, 3, or other number of cardiac cycles, the multiple velocity values may be aggregated (e.g., averaged) over an entire cycle, by starting the injection at different points of the cycle. The averaged value of the blood velocity may represent a relative cleaner signal than a single measurement performed at any arbitrary point in the cardiac cycle, as it may be less strongly dependent, or, in some embodiments, independent of the exact injection times.

In some embodiments, synchronization to ECG signals may be omitted, and measurements may be repeated irrespective of the timing for a sufficiently large number of times to obtain a sufficiently low noise. This may be the case, for example, if the random repetition generates injections at different portions of the cardiac cycle so that each portion of the cardiac cycle of the same length (e.g., each 50 msec of the cycle) is sampled about the same number of times.

Optionally, parameters of the electrodes and/or the bolus are selected such that the time that took the bolus to go from one electrode to another electrode is within the selected portion of the ECG cycle.

It is noted that the injection time may be selected by consideration of a tradeoff of requirements that contradict one another. On one hand, the injection time should be as short as possible, for injection of the bolus volume that is as compact as possible, in order to obtain a graph of voltage versus time which has a drop (or peak) as steep as possible, to provide an indication of the time of the maximum drop (or peak) as accurately as possible. On the other hand, at the same time, the injected bolus volumes should be as large as possible, so that the drop (or peak) in the voltage measurements sensed by the electrodes is sufficiently large to enable accurate detection of the drop (or peak) and corresponding time, even after considerable smearing. While the parameter values described herein provide optimal tradeoffs under tested conditions, it should be understood that other values may be used to provide optimal tradeoffs depending on the actual environment, for example, the bolus size that the bolus injector is capable of providing, the blood vessel diameter, the sheath diameter, the catheter diameter, and the composition of the injected fluid bolus.

At 110, voltage measurements are received, e.g., by computing device 202. The voltage measurements are measured at the electrodes of the catheter, including the first (i.e., proximal) and second (i.e., distal) electrode(s).

Optionally, each electrode measures the voltage continuously, for example, outputting analogue values which may be converted into digital form for further processing.

The injection of the bolus creates a voltage drop (when the bolus of fluid has higher conductivity than the blood) or buildup (when the bolus of fluid has lower conductivity than the blood). The bolus of fluid advances via the flowing blood along the length of the catheter, where electrode captures an indication of the drop or buildup of voltage due to the passing bolus of fluid. The time corresponding to when the local minimum or maximum value of the voltage is sensed by each respective electrode indicates when the bolus of fluid passed by the respective electrode, enabling computation of the velocity of the blood, as described herein.

It is noted that the use of voltage measurements described herein may be used for computing impedance values, optionally by combining them with current measurements. For example, the impedance of a medium between two electrodes may be computed by dividing voltage between the electrodes by current between the electrodes. So wherever voltage measurements are used herein for estimating blood velocity, they may be replaced by impedance measurements, including measurements of real part, imaginary part, magnitude, and/or phase of impedance.

The voltage measurements at the first (i.e., proximal) and second (i.e., distal) electrode(s) are obtained at least when the bolus of fluid flows by them.

The voltage measurements may be continuous and/or discrete in time, capturing the voltage before, during, and/or after the bolus of fluid has passed in proximity to each electrode.

Optionally, at least one of the voltage measurements is a measurement of voltage between two electrodes of the catheter. Alternatively, each one of the voltage measurements is a measurement of voltage between two electrodes (e.g., predefined pair) of the catheter. Alternatively or additionally, at least one of the voltage measurements is a measurement of voltage between a body surface electrode and an electrode of the catheter.

At 112, the voltage measurements are analyzed, for example by processor 204 executing instructions stored on memory 206 using input from data storage device 208.

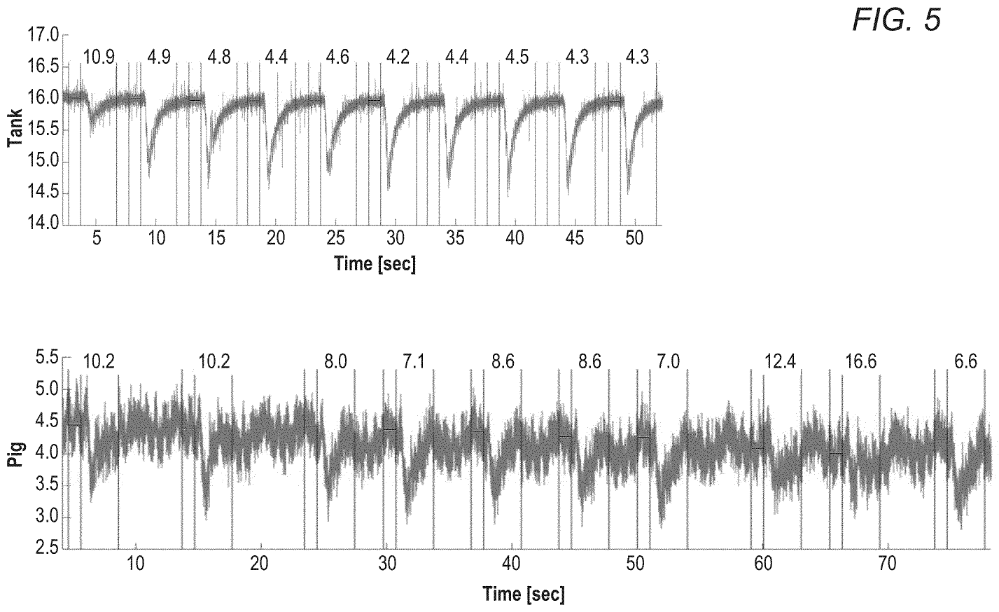

In practice, the measured voltage values have a local drop (or peak) indicating the bolus of fluid. Examples are provided in FIG. 5, showing voltage drops measured in vitro, in a tank (upper graph), and in vivo (lower graph). As the bolus is carried by the blood in the blood flow direction, the bolus stretches out in length, resulting in the appearance of a drop (or peak) becoming smeared in subsequent electrode measurements by increasingly more distal electrodes. The voltage measurements may be analyzed to detect the peak or drop within the smear, for example, using existing peak detection methods and/or cross correlation between two peaks of voltages measured by two of the electrodes. It is noted that cross correlation may improve accuracy of detection of the peak most indicative of the presence of the bolus of fluid, since there may be multiple local peaks generated by factors other than the bolus, for example, the injector and pressure applied to the sheath and/or catheter during its course within the body to the blood vessel (e.g., muscle squeezes, pulsatile pressure of blood vessel walls).

Cross correlation may be performed for the voltage measurements obtained by the two or more electrodes, to identify the peak (or drop) in the electrodes after the first electrode. The peak or drop may be identified in the voltage measurement using standard methods, for example, finding a local minimum or maximum of the voltage measurements. Since it is assumed the bolus has not yet been fully smeared, the peak or drop may be found relatively easily. Once the bolus has travelled along the blood vessels, it becomes smeared, and as a result, the peak or drop may be more difficult to detect for more distal electrodes. The voltage measurements of the more distal electrodes may be cross correlated with the voltage measurements of the first proximal electrode (or to any other less distal electrode) to find the peak or drop in the voltage measurements of the more distal electrodes. In some embodiments, only cross-correlation is used for depicting the movement of the peak in time from the first electrode further.

The voltage measurements may be analyzed for detecting the peak or drop, by detecting a local maximum voltage value in readings of each electrode. The peak may be indicative of a time when the largest volume of the bolus passed in proximity to the respective electrode. As used herein, the term peak may refer to a local minimum value, for example, when the local minimum of a certain measured electrical property is indicative of a time when the largest volume of the bolus passed in proximity to the respective electrode.

At 114, a passage time that took the bolus to go from the first electrode (i.e., proximal) to the second (i.e., distal) electrode is estimated.

Optionally, the passage time is estimated by comparing voltage measurements made at the first and second electrodes. The passage time may be estimated as the difference between the time corresponding to the peak at the first electrode and the time corresponding to the peak at the second electrode.

The bolus injection may be synchronized with a selected portion of the cardiac cycle, for example, synchronized with a certain portion of the ECG signal, as described herein. If the bolus is short enough to go by the two electrodes within the selected portion of the cardiac cycle, the computed passage time (and with it the corresponding blood velocity, estimated at 116, described below) may be associated with the selected portion of the cardiac cycle.

Thus, in some embodiments, the passage time may be significantly short, conceptually corresponding to an instantaneous velocity measurement.

At 116, the velocity of the blood is estimated based on the estimated passage time (as described with reference to 114) and a distance known to exist along the catheter between the first (i.e., proximal) and second (i.e., distal) electrodes. The velocity is computed by dividing the known distance between the at least two electrodes and the time it took for the bolus of fluid to travel between the at least two electrodes.

The velocity of blood may be defined in units of distance per time along the blood flow direction.

Optionally, additional values are determined. For example, a blood flow defined in units of volume per time, denoting the volume of blood that flows in the blood vessel per unit time (e.g., per second) may be determined. It is noted that as used herein, the terms velocity (and flow velocity) denote different entity than flow (and flow rate). The former denoting how fast blood moves from point to point along the catheter, and the second denoting how much blood passes appoint in a second (or other time unit). Flow rate may be measured, for example, by injection of a fluid that changes the impedance of the blood (e.g., saline) to perturb the impedance of the blood near the injection point, and following the decay of the perturbation with time. Thus, the data used for computing the blood velocity is also useful for computing blood flow rate, but for flow rate computation the required input includes a decay rate of a signal received at a single electrode, while for measuring the velocity, passage time between two electrodes is required as input, according to embodiments of the present invention.

A diameter of the blood vessel may be determined based on the estimates of the blood velocity and blood flow, for example, based on known mathematical relationships between flow rate, cross sectional area of the blood vessel, and velocity.

At 118, one or more features described with reference to 104-116 may be iterated.

Time resolution may be increased by combining velocity measurements at different times.

The iterations may be performed by repeating the same injection parameters, optionally at the same injection location. A respective velocity value may be calculated for each injected fluid volume. The multiple velocity values computed for the multiple injections may be analyzed to obtain a resulting velocity value, for example, by averaging the different velocity values. Using the multiple velocity values, each estimated from data obtained at a respective injected volume may provide a more accurate final velocity value than may be provided by a single measurement, for example, by smoothing out noise fluctuations.

In some embodiments, the iterations injection parameters may vary between iterations. For example, the injections may be of different bolus volumes, different injection rate, and/or synchronized to different portions of the cardiac cycle. Each injected bolus fluid may be independently followed as it progresses along the electrodes and analyzed, as described herein, to obtain a corresponding velocity value. The multiple velocity values computed for the multiple injections using different injection parameters may be analyzed to obtain a resulting velocity value, for example, by averaging the different velocity values. Using the multiple velocity values using respective injection parameters may provide a more accurate final velocity value, for example, by smoothing out noise fluctuations.

In some embodiments, the goal of the measurement may include obtaining an average blood flow characterizing an entire cardiac cycle. Thus, if the bolus does not expand along a single cardiac cycle, for example, the passage time is longer or shorter than the time of a heartbeat, it may be desirable to average over measurements taken with boluses that were synchronized with different parts of the cardiac cycle. For example, if the passage time is 1.2 seconds, the heartbeat rate is 60 beats per second, the passage time provides an estimate of the blood velocity during 1.2 heart beats. Since the velocity during the first 0.2 cycle (from the beginning of the bolus) may be different than the average velocity over an entire cycle, the estimate may include an error that depends on the point along the cardiac cycle, with which the bolus is synchronized. Injecting different boluses at respective different points along the cardiac cycle may reduce this error. The same logic applies if the passage time is shorter than a heartbeat.

In some embodiments, the iterations may be performed at different locations within the blood vessel, for example, distal to the stenosis and proximal to the stenosis.

In some embodiments, the iterations may be performed to estimate the velocity of blood at selected portions of the cardiac cycle. For example, each bolus may be injected into the portion of the blood vessel during a different selected portion of the ECG cycle. The velocity of blood may be estimated for each selected portion of the ECG cycle, to provide different estimates of blood flow velocity to different selected ECG cycle portions.

At 120, a diagnosis is determined and/or the patient is treated.

The patient may be diagnosed according to the velocity of the blood, for example, by comparing the estimated velocity of blood proximal to a stenosis with the estimated velocity of blood distal to the stenosis. The patient may be diagnosed according to the diameter of the blood vessel measured using the blood velocity estimates obtained according to embodiments of the present invention, for example, by comparing the measured diameter to a historical measurement and/or to a threshold.

A larger than a threshold difference between the blood velocities estimated proximally and distally to a stenosis may indicate clinically significant stenosis. In another example, the estimated velocity of blood at a certain location is compared to a defined threshold. A velocity value below the defined threshold may be indicative of a clinically significant problem. A significant decrease in diameter and/or a diameter below the threshold may be indicative of clinically significant stenosis that requires treatment.

The patient may be diagnosed, for example, with a clinically significant narrowing of the blood vessel.

The patient may be treated based on the diagnosis and/or based on the estimated velocity. The patient may be treated to expand the diameter of the blood vessel, for example, by insertion of a stent into the narrowing of the blood vessel, balloon expansion of the narrowing, and/or other approaches. The patient may be treated when the patient is diagnosed with a narrowing of the blood vessel.

Reference is now made to FIG. 1B, which is a flowchart of a method of estimating velocity of blood, flowing along a blood vessel at a blood flow direction, utilizing a medical implement that resides in a portion of the blood vessel and comprises a first electrode and a second electrode, in accordance with some embodiments of the present invention. The method may be performed automatically by a hardware processor(s) executing code stored in a memory. The method described with reference to FIG. 1B may be a basis for, and/or include features of other methods described herein, for example, the method described with reference to FIG. 1A. The method described with reference to FIG. 1B may be implemented by components of system 200 described with reference to FIG. 2A.

At 1002, voltage measurements made at the first and second electrodes are accessed. The voltage measurements are made when a bolus of a fluid goes through the portion of the blood vessel at the blood flow direction, and recorded to a digital memory, for example, data storage device 208 and/or measured values repository 208A. The measurements may be accessed in real time and/or later, e.g., after the procedure is over. In some embodiments, the method of FIG. 1A is carried out by a computing device 202 that is not connected to the catheter. In some embodiments, the computing device is connected to the catheter. In some embodiments, the input device is not connected to the catheter but configured to be connected thereto (e.g., by having suitable connectors).

At 1004, the voltage measurements made at the first electrode are compared to the voltage measurements made at the second electrode. The comparison may be performed, for example, by cross-correlation methods to detecting a peak or drop, and defining the time corresponding to the peak or drop at the first and second electrodes.

At 1006, a time that took the bolus to go from the first electrode to the second electrode (which may be referred to herein as passage time) is estimated based on the comparison, for example, computing the difference between the time corresponding to the peak or drop of the second electrode and the time corresponding to the peak or drop of the first electrode.

At 1008, the velocity of the blood is estimated based on the estimated time and a distance known to exist along the medical implement between the first and second electrodes.

Reference is now made to FIG. 1C, which is a flowchart of a method of treating a patient based on estimating velocity of blood flowing in a blood vessel, in accordance with some embodiments of the present invention. The method may be performed, for example, by a physician. The method described with reference to FIG. 1C may be based on, and/or include features of other methods described herein, for example, the method described with reference to FIG. 1A or FIG. 1B. At least a portion of the method described with reference to FIG. 1C may be implemented by components of system 200 described with reference to FIG. 2A.

At 1102, a patient is selected, for example, as described with reference to 102 of FIG. 1A. At 1104, the distal portion of the medical implement, the electrodes, and the aperture for injection of the bolus fluid are positioned, for example, as described with reference to 104 of FIG. 1A.

At 1106, synchronization data is obtained, for example, the physician places ECG electrodes on the patient to obtain ECG data. For example, as described with reference to 106 of FIG. 1A.

The physician may select, e.g., via user interface 224, which portion of the ECG signal to synchronize with the injection. In some embodiments, the physician may select a procedure to inject a series of bolus injections synchronized with different portions of ECG cycle, to allow averaging them to obtain an estimate of the blood velocity over an entire heart beat or an integer number of heart beats. In some embodiments, the physician may select a procedure to repeat injecting short boluses repeatedly at the same portion of the ECG cycle so as to obtain an estimate of the blood velocity at that portion of the ECG cycle.

At 1108, the physician sets the device to inject the bolus of fluid, for example, as described with reference to 108 of FIG. 1A. The device may automatically inject the bolus, or the physician may manually inject the bolus (e.g., by attaching a fluid filled syringe to the sheath, and pressing the plunger of the syringe).

At 1110, the estimated velocity of the blood is computed and presented on a display for viewing by the physician, for example, as described with reference to 110-116 of FIG. 1A.

At 1112, the physician may repeat one or more of 1104-1110, for example, as described with reference to 118 of FIG. 1A.

At 1114, the physician diagnoses and/or treats the patient, for example, as described with reference to 120 of FIG. 1A.

Various embodiments and aspects of the present invention as delineated hereinabove and as claimed in the claims section below find experimental support in the following examples.

### EXAMPLES

Reference is now made to the following examples, which together with the above descriptions illustrate some implementations of the systems, methods, apparatus, and/or code instructions described herein in a non-limiting fashion.

The first set of experiments described below were performed using a model that simulates blood flow in a patient, by simulating blood vessels using synthetic tubes, and using a pump to simulate the heart. The second set of experiments described below were performed on a living pig, to compute blood flow within a coronary artery of the pig.

The experiments were designed to model measuring velocity in the LAD artery. Different blood flow rates were set (i.e., simulated by a pump) to simulate different clinical stenosis conditions in the LAD. Flow rate is expected to be in the range of 2.5-5 ml/s, since severe coronary stenosis is diagnosed with a flow rate of about 2.5 ml/s; and mild stenosis is diagnosed at a flow rate of about 4.8 ml/s (when FFR>0.8).

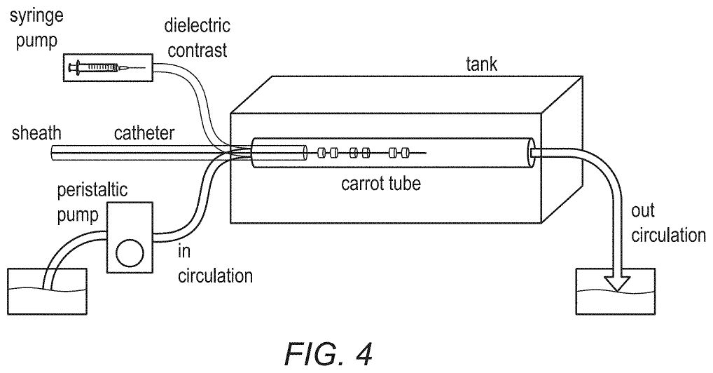

Reference is now made to FIG. 3, which is a schematic of a distal end of a catheter used to measure blood velocity in the coronary arteries of a pig during in vivo experiments, in accordance with some embodiments of the present invention. The same catheter has also been used in the in vitro experiments. Catheter 300, which may be an implementation of medical implement 216, is covered in part by sheath 302, which may be an implementation of sheath and/or tube 236. A contrast agent is injected into the space between sheath 302 and inner catheter 304. Going out of the sheath through an aperture 305 in the sheath, the contrast agent flows with blood (or with a saline solution in the in vitro experiment), and goes along the catheter for about 35 mm to 50 mm (in different embodiments), until it meets the first ring electrode 306, which may be an implementation of electrode 214, and flows along the catheter, going through the 6 ring electrodes. In the shown embodiments, the ring electrodes are arranged in pairs, with 2 mm distance between members of a same pair and 6 mm distance between pairs. Each ring electrode is 0.6 mm in width. All distances between electrodes are measured between electrode centers. It is noted that in operation, the catheter 300 is inside a blood vessel (not drawn). In the in vitro embodiment depicted in FIG. 4 below, the blood vessel was simulated by a carrot, cut to have an inner cavity all along.

Reference is now made to FIG. 4, which is a schematic of an experimental setup for evaluating an in vitro blood velocity estimation process. In this setup, blood was simulated by a 0.9% saline solution, and the dielectric contrast agent was simulated by a 3% saline solution. The blood flow was simulated by flowing the 0.9% saline solution via a peristaltic pump, and the bolus of dielectric contrast agent was generated by a syringe pump. The blood vessel was simulated by a carrot, scooped to have a longitudinal cavity. The distal end of the catheter, described in FIG. 3 was inside the cavity in the carrot.

Reference is now made to FIG. 5, which shows graphs depicting flow measurements received during the in vitro experiment (upper graph) and the in vivo experiment (lower graphs). The Y-axis of both graphs show impedance values.

In the upper graph, each bolus results in a sharp drop in the impedance, followed by a relaxation back to the base-value. In some embodiments, the flow of the blood-simulating liquid (i.e., the 0.9% saline solution) in volume/time can be measured as a ratio between the area between a peak and the baseline. The impedance of the fluid injected in the bolus was lower than that of the blood simulating fluid by about an order of magnitude. Exemplary fluids include 3%, 5%, or 7% saline. In FIG. 5, the flow value (in ml/sec) calculated from each bolus is printed above each respective impedance drop and recovery. These flow values were calculated based on the area between the deep in the graph and the baseline, marked for each respective bolus as a horizontal line.

The lower graph shows similar results obtained from an in vivo experiment, where the blood vessel was an actual artery of a porcine. Here also, each bolus is measured as a deep in the impedance, although the peaks are less sharp.

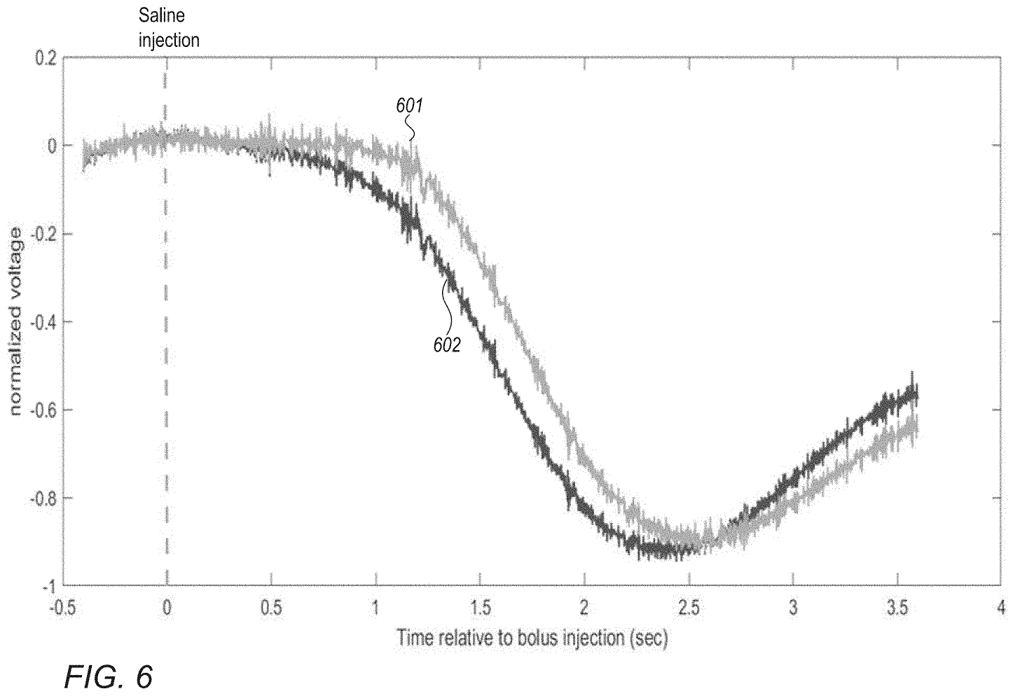

Reference is now made to FIG. 6, which are graphs depicting time development of voltage on two electrodes during a single bolus in the settings depicted in FIG. 4. As the injected current was stable, these voltage measurements were considered equivalent to impedance measurements. Line 601 shows the voltage developed between a first electrode and the ground, and line 602 shows the voltage developed on a second electrode, proximal to the first, and the ground. The ground was the most proximal electrode on the catheter. The space between the two lines is due to the distance that the bolus had to travel from the proximal electrode to the distal electrode. Thus, cross-correlating the two signals, 601 and 602 may reveal the time shift required for the two signals to overlap as closely as possible. This time shift may serve as an estimate to the passage time, i.e., the amount of time required for the bolus to go from the proximal to the distal electrode. When the distance between the electrodes is divided by this passage time, an estimate of the blood velocity is obtained, according to some embodiments.

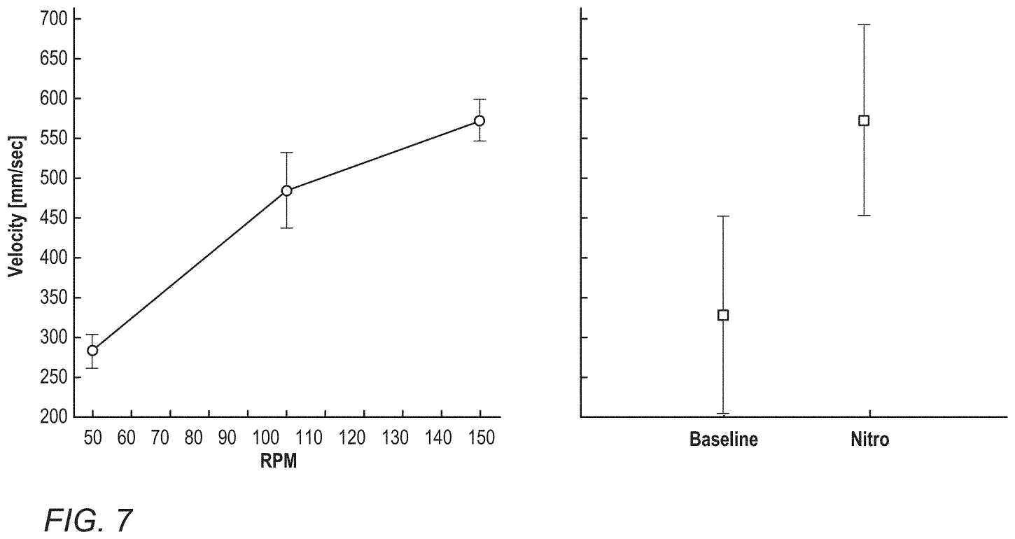

Reference is now made to FIG. 7, which includes a graph depicting change of dielectric contrast agent velocity in a tank due to different pump rates (on the left), and another graph depicting changes in blood velocity measured in a porcine before and after administration of nitroglycerine into the porcine artery, via the catheter.

The graph on the left confirms that higher “blood” velocities are simulated by higher pumping rate of the saline by the peristaltic pump. The graph on the right confirms that nitroglycerin enhances blood rate in the porcine in a degree that can be distinguished by the presently disclosed method. It is noted that the number of repetitions in the in vivo experiment was small, so the error bars are large, but even given these large error bars, the blood velocity after administration of nitroglycerin is significantly higher than before said administration.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant bolus fluids and electrodes will be developed and the scope of the terms bolus fluids and electrodes are intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of estimating velocity of blood, flowing along a blood vessel at a blood flow direction, based on measurements made using a medical implement that resided in a portion of the blood vessel and comprised a first electrode and a second electrode, the method comprising:
accessing voltage measurements measured at the first and second electrodes when a bolus of a fluid went through the portion of the blood vessel at the blood flow direction; wherein the voltage measurements were made at the first and the second electrodes;
estimating a time that took the bolus to go from the first electrode to the second electrode based on the accessed voltage measurements;
estimating the velocity of the blood based on the estimated time and a distance known to exist along the medical implement between the first and second electrodes; and
diagnosing, according to the velocity of the blood, a narrowing of the blood vessel requiring treatment with a stent.

2-3. (canceled)

4. The method of claim 1, wherein at least one of the voltage measurements is a measurement of voltage between two electrodes of the medical implement.

5. The method of claim 1, wherein at least one of the voltage measurements is a measurement of voltage between a body surface electrode and an electrode of the medical implement.

6-8. (canceled)

9. The method of claim 1, further comprising:
obtaining a rough estimation of the blood flow rate, and controlling a bolus injector to inject the bolus into the blood vessel at a flow rate equal to the obtained rough estimation multiplied by a factor between ½ and 2.

10. The method of claim 9, wherein the rough estimation of the blood flow is non-invasively estimated using an external sensor.

11-12. (canceled)

13. The method of claim 1, wherein the velocity of blood is defined in units of distance along the blood flow direction per units of time.

14. The method of claim 1, wherein the bolus of fluid is synchronized for injection into the portion of the blood vessel during a selected portion of an ECG cycle.

15. The method of claim 14, wherein the selected portion of the ECG cycle is selected to encompass a time that takes the bolus to go from the first electrode to the second electrode ECG cycle.

16. The method of claim 14, wherein the velocity of blood is estimated from a plurality of boluses of fluid, each injected into the portion of the blood vessel during a different selected portion of the ECG cycle.

17. The method of claim 14, wherein the velocity of blood is estimated from a plurality of boluses of fluid, each injected into the portion of the blood vessel during a different portion of the ECG cycle, and a velocity of blood is computed based on respective passage times computed for each of the plurality of boluses, each passage time being time estimated to elapse between the bolus passing near the first electrode and the bolus passing near the second electrode.

18. The method of claim 16, wherein for at least one of the boluses, the time that took the bolus to go from the first electrode to the second electrode is a non-integer number of heart beats.

19. The method of claim 14, comprising providing different estimates of blood flow velocity to different selected ECG cycle portions.

20. The method of claim 1, further comprising:
determining a blood flow defined in units of volume per time; and
determining a diameter of the blood vessel based on the estimate of the velocity of the blood and the determined blood flow.

21. (canceled)

22. The method of claim 1, further comprising inserting a stent into a narrowing of the blood vessels detected according to an analysis of the velocity of the blood.

23. (canceled)

24. The method of claim 1, wherein a sheath along the medical implement includes at least one aperture for injection of the bolus of fluid, the at least one aperture located proximal to the electrodes such that in use blood flowing at the blood flow direction passes first near the aperture and then near the electrodes.

25-26. (canceled)

27. A system for estimating velocity of blood, flowing along a blood vessel at a blood flow direction, utilizing data received via a catheter that resided in a portion of the blood vessel and comprised electrodes, the system comprising:
at least one hardware processor executing a code for:
controlling a bolus injector to inject a bolus into the portion of the blood vessel during a selected portion of an ECG cycle obtained from an ECG device that outputs an indication of an ECG signal obtained from ECG sensors sensing the patient;
accessing voltage measurements made at the electrodes when a bolus of the fluid goes through the portion of the blood vessel at the blood flow direction;
comparing the accessed voltage measurements made at the electrodes to obtain an estimate of a time that took the bolus to go from a first one of the electrodes to a second one of the electrodes;
providing the estimate based on the comparison and a distance known to exist along the catheter between the first and second electrodes.

28. (canceled)

29. The system of claim 27, further comprising a bolus injector set to inject the bolus into the blood vessel.

30. The system of claim 27, further comprising an ECG device that outputs an indication of an ECG signal obtained from ECG sensors sensing the patient, wherein the bolus of fluid is synchronized for injection into the portion of the blood vessel during a selected portion of an ECG cycle.

31. The system of claim 30, wherein the ECG device outputs the indication to the at least one hardware processor or to a memory accessible by the at least one hardware processor, and the at least one hardware processor controls the injector based on signals received from the ECG device.

32-34. (canceled)

35. The system of claim 27, further comprising a current source to apply a current to at least one of the electrodes, an ampere meter for measuring the resulting current between the electrodes, and a voltmeter for measuring voltage between the electrodes when the current is applied.

36-37. (canceled)
